(19) United States
(12) Patent Application Publication
(10) Pub. No.: US 2002/0192269 A1
(43) Pub. Date: Dec. 19, 2002

(54) Care system for cosmetic treatment of the skin

(76) Inventor: Ursula Beyer, Bergholz-Rehbrucke (DE)

Correspondence Address: CANTOR COLBURN, LLP, 55 GRIFFIN ROAD SOUTH, BLOOMFIELD, CT, 06002

(21) Appl. No.: 10/114,348

(22) Filed: Apr. 2, 2002

(30) Foreign Application Priority Data
Apr. 10, 2001 (EP) 01108892.9

(51) Int. Cl. A61K 9/70; B32B 27/12

(52) U.S. Cl. 424/443; 442/123

(57) ABSTRACT

The invention consists of a care-system for the cosmetic treatment of skin through layering of a care-material-containing surface element on the skin portions to be treated, at least inclusive of a textile surface, which is compressed, essentially liquid-free, as well as a liquid, wherein the textile product and/or the liquid care-material are exhibited and the textile product for absorption of liquid is expandable to a care material-containing surface element. The care system of the invention demonstrates that through a simple and hygienic application that it can be applied with little time consumption.

The invention consists of a care system for cosmetic treatment of the skin.

Many various state of the art products for skin care are known. Mostly they are creams, ampoules or facemasks of different types that are mostly used to moisturize the skin and impart elasticity.

Currently known available facemasks generally consist of creams or pastes which are spread on the skin and then act to care for it. Its disadvantage is that the dose administration of the masks is found to be difficult. So, often too large an amount of the care treatment product is applied, wherein the excess in conjunction with the treatment must be wiped away or washed off after each treatment depending on the mask used. That will generally feel unpleasant since a greasy feeling remains on the skin and in principle the danger exists that the masks will get into the hair or clothing. Also, adjuvant such as cloth or cotton must often be used particularly for the removal of greasy products. Also, the application of masks on the skin proves to be complicated since the cream or paste-like masks must be applied to the skin with the hands, wherein with face masks the eye areas and the mouth are largely left out, whereas the eye area is particularly sensitive and as a rule requires a different care treatment than the rest of the skin. This is complicated and can only proceed with the aid of a mirror, as otherwise the hazard exists that the mask will be applied upon the irritable skin portions or will not be uniformly spread out upon the treatment areas. Besides this inconvenience, residue of the mask remains on the hands and this will be smeared lightly on the hair, on the clothing or on the instruments. Therefore, the traditional masks cannot be applied in fast and uncomplicated intervals, but on the contrary require a definite time expenditure and they cannot be quickly, and, above all, cleanly applied. Therefore, the user must utilize a different time period for skin care, which is not always possible and has the consequence that the skin is often cared for irregularly.

A further problem pertains to the state of the art of known care products, that the danger exists that they can become contaminated and consequently have damaging effects especially on the sensitive parts of the skin. Therefore, skin inflammations, rashes and other skin irritation result. Also, bacterial infections are possible. This leads back to the fact that the care products for the most part are sold in large tubes which are often not closed correctly. Also, residue remains on the tube opening with routine handling which can become contaminated and get upon the skin with the next application. The opened products have only a limited stability, so that opened tubes must be attended to after a time, which is unfavorable to the user on a cost basis. Nevertheless, the user then returns several more times to the opened products based on his cost awareness when, for hygienic reasons, he should no longer be using them. Also, it is often not quite clear how long may the open product be used after it has been opened, which represents a substantial uncertainty factor. The resulting skin irritations are then related to the use of the product, which causes a loss of image and will result in economic damage to the manufacturer.

There are solution to avoid this problem. Thus, it is known under the state of the art, for example, the care products can be portioned by packaging these products in ampoule size units. Although, this variation avoids the hygiene problem, but it has the disadvantage in the difficulty of application, which still results with the introduction of the care material on the skin with the hands and with the liquid care materials the danger also is present that the liquid drips on the clothing which has in addition to the undesirable spots, the further disadvantage of wasting care product.

Therein lies the problem, which is the basis for the present invention, in making available a care system which tends to improve substantially the state of the art since it is simple and fast in its application.

This problem is solved by a care system for the cosmetic treatment of skin by a superimposed layer of care material-containing surface elements on the skin portions to be treated, which essentially make the product liquid free by compressing it, as well as a liquid, wherein the textile product and/or the liquid care material and the textile product contain care materials for absorption of liquid to a care material-containing surface element.

The care system comprises a textile product, which is compressed to become essentially liquid-free. Therefore, the textile product can be packed compactly and takes up only little room besides having only a small surface area. Thereby, the manipulation of textile product is simplified in an advantageous manner and further an extensive soiling of surface area is prevented. In the essential liquid-free, in the sense of the invention, means that the moisture has been drawn away from the textile product, which has, furthermore, been compressed, into a small volume. Thereby is formed a dry compact unit with a small volume.

The care system further comprises a liquid. With the care system of the invention either the liquid and/or the textile product comprise the care material, wherein the care materials are preferably contained in the fluid.

The textile product is expandable to a care material-containing element after absorption of the fluid. In order for the compressed textile product to contain a care material-containing surface element, the compressed textile product is brought into contact with the fluid of the present invention system. The liquid is taken up by the essentially liquid-free compressed textile product. In view of the liquid absorption into the web textile products, these expand and abandon their compressed form. The liquid-soaked textile product is ready to use in the expanded condition and can be spread out by unfolding as a care material-containing surface element. The care material-containing surface element can be applied in the expanded state to the skin portions to be treated where the care material can then act directly upon the skin, thereby treating same. Thus, since the liquid part of the care material is taken up by the textile product surface, the textile product in the water-soaked state forms a care material soaked tissue mask with care-imparting moisture film. That the compressed textile product is essentially dry has the further advantage for the user that it primarily only absorbs the fluid which contains the care material. Thus, the mask has a large absorption volume and is completely soaked with care material.

Thus, a care material-containing surface element is convertible, that is, for example, unfolded elements can be placed on the skin portions to be treated. Preferably, the care material-containing surface element is pressed lightly on the skin, so as to warrant a good contact between the textile product and the skin. The care material is in direct contact with the skin and settles on it. Thereby is avoided in an advantageous manner, as compared to traditional masks, excessive care material residue remaining on the skin, which must be removed.

The textile product surfaces remain on the skin for a specified period of time and can easily provide for a specified working time. The correct working time period to be used depends upon the care system capacity. For example, care systems can be varied according to the desired effect. The skin can be further directly treated in connection with the treatment, for example is rouged, but first expensive care material residue must be removed. Thus, the care system of the invention is inexpensive since also no separate cloths or cotton pads will be required in order to remove the surplus residue. Surplus residue remains in the textile product and is present together with this. Hence, the care system can easily be used, for instance for travel in an airplane.

The care system of the invention presents many advantages over the current state of the art. It is simpler, cleaner and quick in application. In order to prepare a functioning care system, one must only bring a compressed textile product into contact with the liquid, whereby the textile product can be converted into a care material-containing surface element by the liquid absorption. This, for example, can take place by unfolding of textile product to form a care treatment textile mask. Advantageously, it is not necessary for the care of the skin to distribute with the fingers a cream or a paste upon the face. Thereby, the invention is advantageous in circumventing the need to cleanse the hands after the care, since for the user of care systems only a minimal contact with the hands is necessary, since a large part of care material is bound in the textile product and a care-film forms only on the surface element, so that the care material is essentially left on first by contact with the skin surface to be treated. Thereby is the amount, other than with a cream or paste, always proportionately small, in order that the care system is practically cleaner and the risk of spots is minimized.

Furthermore, the disclosed care system invention is easy to dose. It will prevent in an advantageous way too much or too little care material staying on the skin since only as much liquid is absorbed out of the soaked textile mask as necessary. The care system is quick in handling and is thereby now and then conveniently utilizable to cool and relax the skin. Mirrors or similar articles are not necessary to obtain as textile product supplements for the product application. Therefore, the care system can also be used while traveling.

A further advantage of the care system of the disclosed invention lies therein that it can be used extremely hygienically. For an application, required liquid is brought into contact with the compressed textile product and first the textile product, by the liquid absorption, forms the ready-made mask directly. It is a hidden advantage of a system that the masks are soaked. This will prevent the masks from being dry or soiled, as the textile product in compressed form somewhat attracts dirt. Also, it will prevent altering of the composition of the mask in storage, for example, since water evaporates and therefore the concentration of care material changes. The mask is disposed of after use. In this way, contaminations due to standing or similar effects are prevented since repeated use of the item is not necessary.

Accordingly, an important development is suggested that the textile product and the liquid can be packaged in essentially liquid-free solid form. Thereby, in an advantageous way diluted moisture present on the components of care systems and the damage due to same, for example, in view of untimely swelling of the compressed textile product or through evaporation or contamination of liquid, is prevented. Moreover, a hygienic, such as a sterile package can also be subsumed in the essentially liquid-free solid. Finally, it is recommended that the packaging be so selected that the danger of component contamination and skin irritation due to it is minimized. For the packaging, various packing methods come into consideration, such as sealing or shrink-wrapping, for instance. That the textile product for the contact with the liquid of the care system is in the essentially liquid-free solid form and furthermore is preferably packed in liquid-free solid has obvious above described advantages. However, explanation forms for the invention are also included for the case of textile product slightly bulging. This can, for example, occur by mishandling or too long storage. A small bulge does not alter the basic principle of the care system invention, they are only slightly less advantageous.

Accordingly, a further development has been proposed, that the liquid contains the skin treatment materials and these care materials together with the liquid are absorbable by the compressed textile product, whereas the textile product after absorption of liquid cannot be folded to a care material-containing surface element and the care-material acts by contact of these surface elements with the skin. The application of liquid with care material simplifies the preparation and management of care systems and it further enables a much larger amount of care material to suffice. These procedures can be varied slightly. This accounts for the characteristic that the liquid containing care materials with the flat spread textile product can be varied. That makes it possible to create different cosmetic care masks with the textile product by variation of its component elements and to achieve different care characteristics. Therefore, various skin segments can be effectively cared for.

According to the invention, an advantageous development is proposed, that the product surfaces consist of a natural product, preferably cotton. However, synthetic fabric variations are also possible and lie within the scope of the invention. Preferably, the flat textile product should be made of cotton fleece. That has the advantage that it is absorbent and soft, and can optimally press against the skin as we also feel the absorption.

Accordingly, advantageous textile product savings are another important development. This is an especially great advantage with facemasks, since in the textile product, the eyes and mouth portions can be saved, so that the care material does not act in this area. That will avoid, in a simple way, the necessity of accurate spreading of the creme or paste in facial treatment of the mouth and eye portions which must be particularly spared by a complicated neatness of cream or paste application. This promotes the easy handling of the care system, since it can be used rapidly and without complications. Also, it has been recommended to make incisions alongside the nose surface areas so that the nose area and face are optimally covered and no hollow areas occur under the textile product surfaces.

Thus, a good layer of mask and a narrow skin contact are possible. Also, other forms for the mask are conceivable depending upon which portions should be treated. Therefore, textile products are conceivable which are formed specially for instance, for the eyes or cleavage. These can then be combined with suitable, fitting care products. The system of the invention can thence be fitted on the different body portions.

According to an advantageous development, it is proposed that the shaped compressed textile product is compressed into a compact form wherein the shaped textile product is compressed into a minimal volume. Thus, the textile product takes up only a little room, which moreover conserves packing material. In view of the concentrated form and the essentially liquid-free tight packaging, it is established that the textile product surface is in the essentially liquid-free solid state and due to this packaging can absorb no moisture from the air. Thus it is warranted, that in view of this complete soaking by the contact with the care material-containing liquid a part of the ready absorption potential is not consumed due to care material-containing moisture. Thus, it is made possible that a high concentration of care-treatment agent in the care material-containing surface element is possible. Thus, there are different possibilities wherein compressed compact form is prepared, for instance, by vacuum pump. Thereby the compact forms, for example, can be cube-shaped, tab-formed, round or have rounded edges.

Accordingly, an important development of the present invention is further proposed in that the amount of liquid, which with the compact, compressed textile product surface is brought into contact, is so chosen that the compressed textile product is extended to its full volume and is expandable in the liquid soaked state to form a moist surface element. It is thus warranted that the textile product can fully be unfolded to a surface element and thereby is soaked completely with the liquid and therefore with the care material. In view of a moisture excess is the transfer of a liquid surplus of the liquid film on the skin promoted. This can be expedited by pressure of the unfolded surface elements.

According to an advantageous development, it is further recommended in case of the invention, that liquid and textile product surface compressed to a functional unit, essentially forming a storage container with two functional chambers, wherein one chamber holds the compressed textile product and the other a care material-containing liquid, wherein the tab of the one chamber runs across the liquid containing chamber and the textile product after absorption of liquid is expandable to a textile product containing surface element. Thus, the care system is adaptable in an advantageous way for a hygienic one-time use. By this arrangement a fast, convenient unit is placed at one's disposal, which can be immediately discarded after use. The comprehensive summary of the care systems in a specific arrangement is particularly advantageous for use in transit, such as for travel. The device can be sealed liquid tight, to prevent that moisture penetrates into the chambers. Thereby, the condition of textile product and liquid is conserved until use. This is advantageous, so that the fluid reservoir contains exactly the amount of liquid required to prepare the compressed textile product completely extended and ready-to-unfold.

Furthermore, with the method of the invention for the cosmetic treatment of skin by coating of a care material-containing surface element, the skin is placed under protection, which is thereby characterized such that compressed textile product surface in essentially liquid free solid form is brought into contact with a liquid, wherein the liquid and/or the textile product surface is comprised of the skin caring material, the textile product absorbs the liquid and the textile product in the liquid-soaked state is unfolded to a care material-containing surface element and is laid upon the portion of the skin to be treated. This method can be demonstrated with a care system according to claims 1-9.

Further advantages and characteristics are proven on the basis of the figures and the pertinent description. These serve exclusively as the closest explanations and point out several possible arrangements of invention embodiment care systems. Thereby are designated:

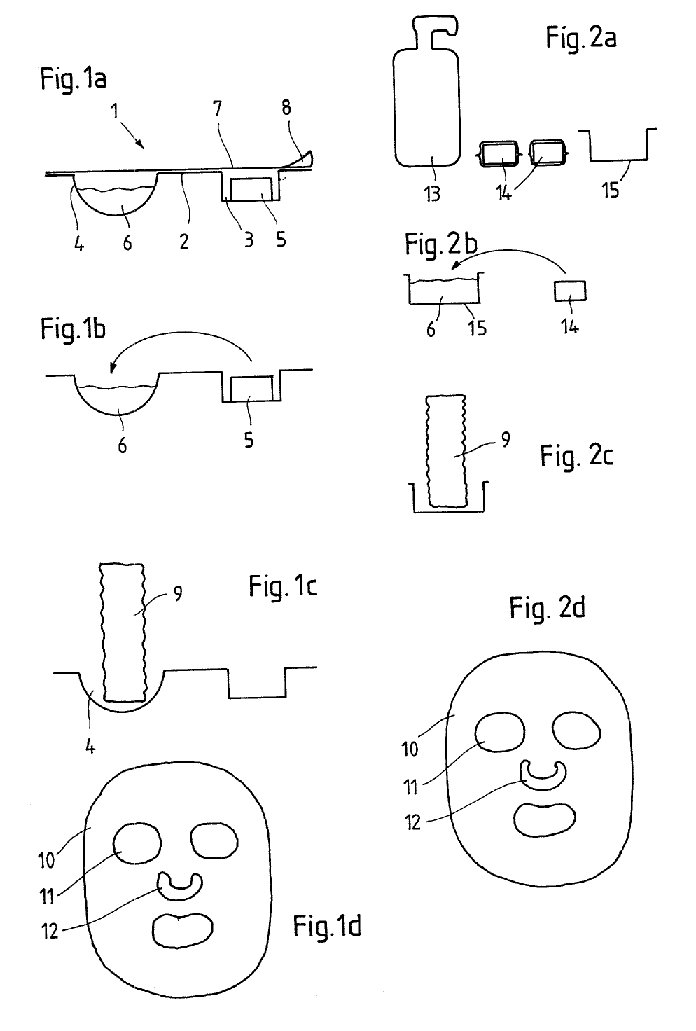

FIGS. 1a-1d the care system of the invention according to a working plan variation, as well as its applications

FIGS. 2a-2d the care system of the invention according to a further working plan variation, as well as applications of these care systems.

In FIG. 1a a care system of the invention in a sealed condition is presented. Therein, a working example for a functional unit 1 is summarized. These consist of a storage container 2 of synthetic material exhibiting two chambers 3 and 4. In the somewhat smaller shaped chamber 3 one finds at a compact tab the compressed textile product 5. In chamber 4 one finds the skin care agent material. In order to protect the compact compressed textile article 5 as well as the liquid 6 from contamination and water, the storage container is sealed with a lid 7. By means of the release closure 8 the lid 7 can easily be removed from the storage container, so that both chambers 3 and 4 lay open. The compressed textile product 5 compact tab is taken out of chamber 3 and goes into the filled chamber 4 (FIG. 1b). By contact with the liquid the tab 5 springs off and the textile product spreads itself out to its normal size 9 through the liquid intake. Thus, it is advantageous if reservoir 4 is directly filled with so much liquid that it is sufficient to change around the compressed textile product 5 in a care material containing surface element 9 which is unfolded (FIG. 1c). With cotton fleece masks which, for example, are especially suited for overlaying on the face areas about 6 ml. is sufficient. But this is dependent upon the size of the mask as well as on useful textile materials and can vary from this. By the exact adjustment of the amount of liquid for the absorption potential, the resulting textile products will be profitably limited since much excessive moisture residue remains in the chamber 4 which, in addition to a waste of care product, would also be less clean, as this residue could leak out. FIG. 1d shows the care material-containing surface element 10 in an unfolded form. The unfolding can be done in simple manner with the thumbs and forefingers. Thus, remaining care material residue remains only on the fingertips of each two fingers, where it quickly soaks in. In the unfolded state, the care material-containing surface element can be applied to the skin portions to be treated. The previous working example deals with a cotton fleece textile product, which in advantageous manner exhibits savings 11. These savings in the mask 10 expose mouth and eye areas of the face and in an advantageous way further support the nose slits 12 so that the textile product 10 can optimally fit itself to the facial contours. The user can easily place the mask on the face near the savings 11 and lightly press on the skin, in order to obtain an optimal contact between the skin and the care material soaked textile mask 10. In view of the savings in 11 it is not necessary with the use of the mask to take special precautions in regard to preventing contact between the care product and the sensitive eye and lip areas. Thus the mask 10 is simple in its use and can also be used fast in between intervals without special precautions necessarily being applied.

FIGS. 2a-2d as well as FIGS. 1a-1d show the care system according to the invention and its use according to a further method variation. According to this method variation one finds the care active liquid 6 in liquid dispenser 13 and the textile tabs are packed separately. The care system is in the form especially suitable for many person households or when frequent care applications take place. In view of the individual packaging the compact compressed textile product 14 is protected from moisture and can be sold in a box together with the liquid dispenser. Thus, it is possible to sell liquids with different care materials, which then can be combined with the textile products 14. So, it is possible with the care system to prepare different working masks so that liquids with different care materials can be available. The care material-containing liquid is given in a container 15. Thereby, this can display direction labels which are necessary for the full utilization of the textile products and which list, for instance, the required amount of liquid dosage to be used. Also, it is conceivable that the pump-dispenser is so regulated that the required amount of liquid can be dose delivered with unparalleled activity. The compact compressed textile product 14 is transported into the container 15 with the care material-containing liquid 6 (FIG. 2b). The textile material expands through the wetness and takes up the care-material into its textile web and is then in the liquid soaked, expanded form 9 (FIG. 2b). This care material-soaked and expanded textile product can be unfolded upon a care material-containing surface element 10 and be applied for the treatment upon the cared-for skin portions. After use these can easily and cleanly be disposed of without danger of the residue being smeared.

The possibility also exists that the textile product contains the care material. For example, this is advantageous if the care material is not damaged by the pressing procedure. By contact of the care material-containing compressed textile products with a liquid such as water, the textile product expands and the care material is dissolved. Thereby, a care material-containing mask is formed, which by unfolding can be spread out upon a surface area, which then can be applied to the skin portions to be treated.

Reference Listing

| | |
|---|---|
| 1 | functional unit |
| 2 | storage container |
| 3 | chamber for textile material |
| 4 | chamber for liquid |
| 5 | compressed textile material |
| 6 | liquid with care materials |
| 7 | lid |
| 8 | opener bottle |
| 9 | care material-containing surface element |
| 10 | unfolded care material-containing surface element |
| 11 | open space |
| 12 | nose slits |
| 13 | liquid dispenser |
| 14 | compressed textile product |
| 15 | container |

1. Care systems for cosmetic treatment of skin by layering of care material-containing surface elements onto the treated skin portions, comprising a textile product which is compressed essentially liquid-free, as well as a liquid, wherein the textile product and/or the liquid care material is expandable.

2. Care system of claim 1, characterized in that the textile product and the liquid are packed essentially leakproof.

3. Care system according to claims 1 and 2, is characterized in that the liquid contains the skin care material and this care material together with the liquid are absorbed by the compressed textile product, wherein the textile product in the liquid soaked state is expandable to a care material-containing surface element and the care material acts upon the skin by contact with the surface element.

4. Care system according to one or more of claims 1-3, characterized in that the textile product is of cotton.

5. Care system according to one or more of claims 1-4, characterized in that the textile products exhibit recesses.

6. Care system according to one or more of claims 1-5, characterized in that the textile product is a compressed compact form, whereby the textile product is compressed to a minimal volume.

7. Care system according to one or more of examples 1-6, characterized in that the liquid amount with which the compressed textile product is brought into contact is so chosen, that the compressed textile product is expanded to its full volume and in the liquid soaked state is expandable to a moistened surface element.

8. Care system according to one or more of claims 1-7, characterized in that the liquid and textile product surfaces are compressed to a functional unit, wherein same consists essentially of a storage container with two chambers, wherein one chamber retains the compressed textile product and the other a care material-containing liquid, wherein the compressed textile product is inserted into the liquid containing chamber and the textile product expands to a care material-containing surface element by absorption of liquid.

9. Care system according to claim 8, characterized in that the arrangement is sealed liquid tight.

10. Method for the cosmetic treatment of skin by layer coating of care material-containing surface elements, characterized in that,
the essentially liquid free compressed textile product is brought into contact with a liquid, wherein the liquid and/or the textile product surface retain the skin caring agent.
the textile product soaks up a liquid
the textile product in liquid soaked state is expanded to a care material-containing surface element and the surface element is applied to the portion of the skin to be treated.
